(19) United States
(12) Patent Application Publication
(10) Pub. No.: US 2018/0253535 A1
(43) Pub. Date: Sep. 6, 2018

(54) Control Center and Licensing Management System for an Intraoral Sensor

(71) Applicant: Todd C. Miller, San Jose, CA (US)

(72) Inventor: Todd C. Miller, San Jose, CA (US)

(21) Appl. No.: 15/901,813

(22) Filed: Feb. 21, 2018

Related U.S. Application Data
(60) Provisional application No. 62/462,238, filed on Feb. 22, 2017.

(51) Int. Cl. G06F 21/10; A61B 6/14; G06Q 10/10

(52) U.S. Cl. CPC G06F 21/105; G06Q 10/10; A61B 6/145

(57) ABSTRACT

A sensor licensing system for an intraoral sensor includes an on/off mechanism, a driver and a sensor. The on/off mechanism is coupled to the intraoral sensor and has an encrypted licensing code so that the on/off mechanism turns on and off the intraoral sensor. The driver processes the encrypted license code stored in the sensor memory and determines if license code is expired based on the computer clock, exposure counter or other desired limits. The sensor periodically checks an online server for an updated encrypted license code and stores new license code in sensor memory. The updated license code is created and stored on the server when a new periodic subscription payment is made. The sensor periodically checks and downloads any updated license code. Failure to make a payment will result in no encrypted license code update being created and eventual expiration of the old encrypted license code.

### BACKGROUND OF THE INVENTION

### Field of the Invention

The present invention generally relates to a licensing management system for an intraoral sensor and particularly to the management of licenses, licensing data, tamper protection, x-ray dose optimization, exposure monitoring and other status/support information.

### Description of the Prior Art

Dentists have used x-rays to image teeth and parts of the mouth for many years. In general, the process involves generating x-rays and directing the x-rays at the patient's mouth. The x-rays are attenuated differently by different parts of the mouth (e.g., bone versus tissue) and this difference in attenuation is used to create an image by using electronic image sensor.

Many commercial transactions are based on the licensing of property, products or services which may involve a limitation of the scope and/or duration of their use. Licensing contracts may operate to restrict or otherwise limit the user's ability to assign, redistribute, resale or otherwise alter the intended beneficiary of the license, while other restrictions may be directed to how, when, where and for how long the use may occur. With the growth of licensing business models, problems involving the efficient distribution, authorized conveyance, tracking, and management of licenses, both by licensees and licensors, has grown as well.

U.S. Patent Application Publication No. 2002/0107809 teaches a license management system for managing licensing data that may be applied to any property, product and/or service licensing model. The licensing management system includes client and server managed security features to control or otherwise monitor and/or restrict the use and re-distribution of licensed subject matter.

Hardware application vendors are faced with several challenges when trying to establish and manage product licensing for their products, such as: managing order entry; tracking product use; offering multiple licensing options; integrating license management into product installation; ensuring licensing information and product source are secure; managing distributed support across a network; customizing the licensing process; giving end-user's control over how their licenses are dynamically distributed and employed and controlling the cost and complexity of license management. There is a need to address these and similar deficiencies associated with the effective and efficient management of licensing in a wide variety of licensee and licensor market environments. With respect to the hardware device industry, a need exists for a turnkey electronic method for obtaining licenses on an as needed basis. A need also exists for a network-based system that allows user's online access to a variety of application tools that may be used on a trial basis to determine product usefulness and then purchased in a secure, convenient fashion for as long as the application product is needed or on a subscription basis.

A method for managing licensing data which U.S. Patent Application Publication No. 2002/0107809 also teaches includes the steps of providing a host system having a processor for processing digital data and providing a client system having a processor for processing digital data and communicably connected to the host system. The method also includes the steps of providing a license management host application running on the host system with the host application having access to a licensing database and providing a license management client application running on the client system with client application having access to a client license datastore. The method further includes the steps of providing a user interface configured to accept licensing orders with the user interface providing a user with access for ordering a license, said user interface requesting the issuance of a license; issuing a license from the licensing database, using the management host application, in response to a user interface request to add a license to the client license datastore, monitoring usage of a license so issued using the management host application, compiling and displaying at least a plurality of licenses stored in the client license datastore using the license management client application and communicating with the host application thereby using the license management client application to modify terms corresponding to at least one license stored in the client license datastore.

The applicants hereby incorporate the above referenced patent and patent application publications into their specification.

### SUMMARY OF INVENTION

The present invention is a sensor licensing system for an intraoral sensor which includes an on/off mechanism, a driver, a sensor and a sensor memory. The on/off mechanism is coupled to the intraoral sensor and has an encrypted licensing code so that the on/off mechanism turns on and off the intraoral sensor.

In the first aspect of the present invention the driver processes the encrypted license code stored in the sensor memory and determines if license code is expired based on the computer clock, exposure counter or other desired limits.

In the second aspect of the present invention the sensor periodically checks an online server for an updated encrypted license code and stores new license code in the sensor memory.

In the third aspect of the present invention the updated license code is created and stored on the server when a new periodic subscription payment is made.

In the fourth aspect of the present invention the control center periodically checks and downloads any updated license code.

In the fifth aspect of the present invention a failure to make a payment will result in no encrypted license code update being created and eventual expiration of the old encrypted license code.

In the sixth aspect of the present invention the encrypted licensing code incorporates date and image counter checking to prevent tampering.

In the seventh aspect of the present invention the sensor licensing system includes a mechanism of detecting potential expiration of sensor license and notifying user of need to either update license or connect sensor to a server to check/update licensing status.

In the eighth aspect of the present invention the driver controls operation of the intraoral sensor on a computer and the software operates on the computer not only in real time, but also in the background so that the control center controls image acquisition and settings for the intraoral sensor so that it can be used with almost any dental imaging software on the market.

In the ninth aspect of the present invention the control center also monitors performance of the intraoral sensor during use and notifies the user of problems.

In the tenth aspect of the present invention the control center acts as the primary driver for the intraoral sensor and interfaces with other imaging applications to acquire and serve up images for processing and display wherein the dental image acquisition software interfaces with other applications via either an SDK driver or a TWAIN driver whereby the control center can perform preprocessing on the image before transferring to an imaging application, including but not limited noise filtering, sharpening, contrast enhancement or histogram equalization.

In the eleventh aspect of the present invention a dose optimization tool for an intraoral sensor guides user to perform initial exposure measurements of an x-ray head for optimal signal level to minimize patient exposure to ionized radiation to insure optimal image quality while insuring that the lowest possible x-ray generator settings to achieve diagnosable x-ray images.

In the twelfth aspect of the present invention the dose optimization tool recommends ideal dose/time settings for use based on adult/child and different x-ray energies.

In the thirteenth aspect of the present invention the dose optimization tool includes a real-time signal monitor which monitors image level and will continue to monitor exposure of every image and notify the operator if signal is too high or low immediately after images acquisition during clinical use.

In the fourteenth aspect of the present invention the dose monitor of the dose optimization tool operates in the background thereby allowing it to be used independent of the imaging application software being used.

In the fifteenth aspect of the present invention the exposure monitor counts sensor usage and records data such as total exposures.

In the sixteenth aspect of the present invention the control center further saves and stores recent images to allow for remote review and diagnosis of sensor issues during debugging.

In the seventeenth aspect of the present invention the control center provides integrated support which is live with built-in support tools operating in the background, independent of the imaging application.

In the eighteenth aspect of the present invention the control center monitors real time for software or sensor firmware updates.

Other aspects and many of the attendant advantages will be more readily appreciated as the same becomes better understood by reference to the following detailed description and considered in connection with the accompanying drawing in which like reference symbols designate like parts throughout the figures.

The features of the invention which are believed to be novel are set forth with particularity in the appended claims.

### DESCRIPTION OF THE DRAWINGS

FIG. 1 is a schematic diagram of a dental x-ray system including an x-ray source, an intraoral sensor located in a patient's mouth and a computer connected to the intraoral sensor according to U.S. Pat. No. 9,259,197.

FIG. 2 is an exploded perspective drawing of an intraoral sensor which has electronic components, both in its head side and its tail side, for operating a control center according to the present invention.

FIG. 3 is a diagram of a control center which manages licenses, licensing data, tamper protection, x-ray dose optimization, exposure monitoring and other information for use with the intraoral sensor of FIG. 2 according to the present invention.

FIG. 4 is an overview of the involved parties required to handle the x-ray sensor license distribution and enforcement in the form of a schematic drawing of a licensing management system of the control center of FIG. 3 which includes a plurality of control centers, a plurality of intraoral sensors, an FTP license server, a license database server with a memory containing licenses, a licensing client and a payment system according to the present invention.

FIG. 5 is a diagram of the interaction of at least one of the control centers, at least one of the intraoral sensors, the FTP license server, a license database server with a memory containing licenses, the licensing client and the payment system of the licensing management system of FIG. 4.

### DESCRIPTION OF THE PREFERRED EMBODIMENT

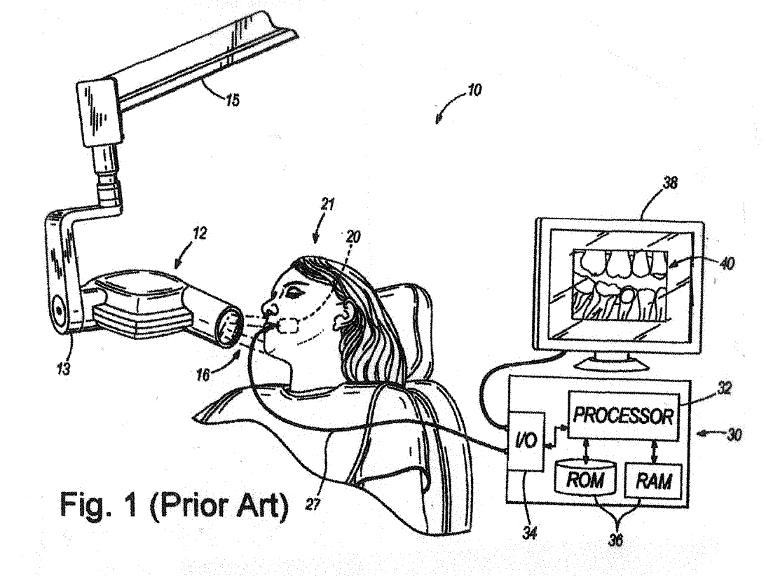

Referring to FIG. 1 a dental x-ray system 10, which U.S. Pat. No. 9,259,197 teaches, includes an x-ray source 12. The x-ray source 12 is located on an end 13 of a mechanical arm 15. When activated, the x-ray source 12 generates an x-ray stream 16 that has a generally circular cross-section. The x-ray source 12 is positioned by an operator so that the x-ray stream 16 is directed to an intraoral sensor 20. The intraoral sensor 20 is placed in the mouth of a patient 21. The intraoral sensor 20 may include a scintillator that coverts x-ray radiation to visible light. The intraoral sensor 20 is configured to convert x-rays into electric charge, which in turn are converted to digital signals. These digital signals are provided to a processor 32 which is connected to memory 36, ROM and RAM, and an input-output interface 34. Image data captured by the intraoral sensor 20 and processed by the computer 30 is sent to a display 38 and viewed as image 40.

Still referring to FIG. 1 in the dental x-ray system 10 capturing an image depends on at least two factors: activation of the x-ray source 12 and “activation” of the intraoral sensor 20. What constitutes “activation” of the intraoral sensor 20 can vary based upon the type of intraoral sensor 20 used, but in most cases “activation” occurs when the intraoral sensor 20 detects the x-ray exposure and then automatically starts image capture which is the integration of the image. It would be advantageous if the automatic “activation” of the intraoral sensor 20 could be prohibited depending on the licensing status

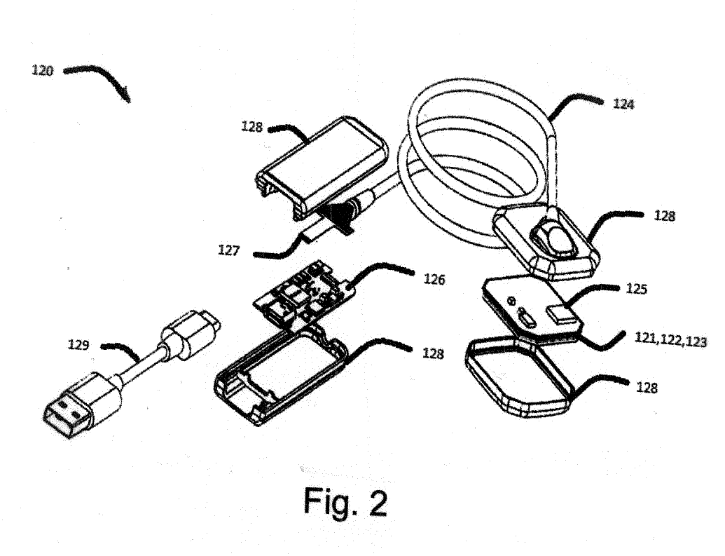

Referring to FIG. 2 an intraoral sensor 120 which includes an imaging sensor 121, a fiber optic 122, a scintillator 121, a sensor printed circuit board (Head side PCB) 125, a multi-conductor cable 124, an interface printed circuit board (Tail side PCB) 126, a connector 127 and housings 128. The imaging sensor 121 converts x-ray energy into digital signals. The multi-conductor cable transmits digital signals from the Head side PCB 125 to the Tail side PCB 126. The Tail side PCB 126 includes electronics that receives digital data from the Head side PCB 125 and transfers it to a host computer USB interface. A Type C USB connector 129 communicates with a host computer via replaceable USB-C to USB-A extension cable. The housings 128 are a set of hermetically sealed, plastic parts that protect sensor components from mechanical and environmental conditions.

Still referring to FIG. 2, the major components in the Tail side PCB consists of USB controller IC, a FPGA, a flash memory and power regulation circuits. In the sensor head electronics, digital imager data is serialized and is sent to the Tail board. Communication and data transfer from the sensor to the host computer is handled by a controller chip located in the tail PCB and handles communication via a USB protocol. Some/all functions located in the Tail Side may alternatively be housed on the Sensor Head.

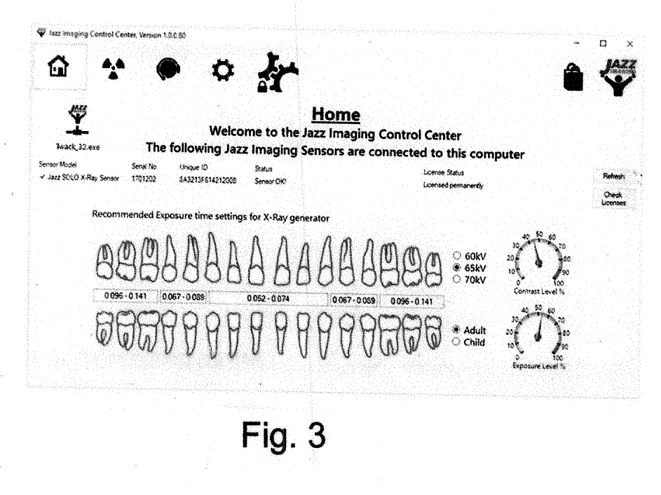

Referring to FIG. 3 in conjunction with FIG. 2 a control center 520 manages licenses, licensing data, tamper protection, x-ray dose optimization, exposure monitoring and other information for use with the intraoral sensor 120. The control center for dental imaging operates in both real time and the background in order to control image acquisition and settings for the intraoral sensor 120 so that it can be used with almost any imaging software on the market. The control center 520 for dental imaging also monitors the sensor performance during use and notifies the user of problems. Image acquisition software acts as the primary driver for the intraoral sensor 120 and interfaces with other imaging applications to acquire and serve up images for processing and display. The interface with other applications can either be direct using a software development kit (“SDK”) or using a TWAIN data source. The software can perform pre-processing on the image before transferring to the imaging application, including noise filtering, sharpening, histogram equalization.

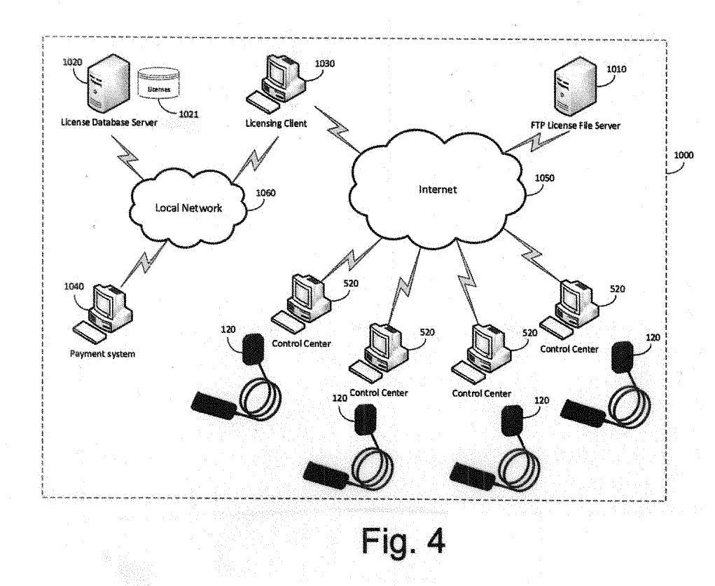

Referring to FIG. 4 in conjunction with FIG. 2 and FIG. 3 the control center 520 includes at least one intraoral sensor 120, at least one control center 520, an FTP license server 1010, a license database server 1020 with a memory 1021 containing licenses, a licensing client 1030 and a payment system 1040. These are the involved parties required to handle the x-ray sensor license distribution and enforcement.

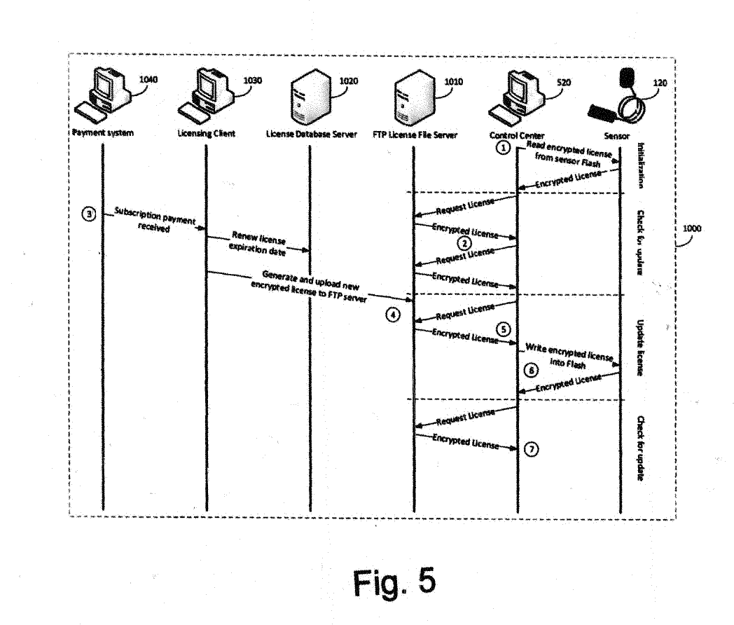

Referring to FIG. 5 in conjunction with FIG. 4 the interaction of at least one intraoral sensor 120, at least one control center 520, the FTP license server 1010, the license database server 1020 with a memory 1021 containing licenses, the licensing client 1030 and the payment system 1040 is as described below. The FTP license file server 1010 has a processor and license file storage coupled to the processor. When the intraoral sensor 120 is connected to the computer, the control center 520 reads the encrypted license from the flash memory. The intraoral sensor 120 is switched ON or OFF depending on license contents. When the control center 520 reads the license from the intraoral sensor 120, the control center 520 periodically requests encrypted licenses from the FTP license file server 1010 and determines whether it has been updated compared to the license read from the intraoral sensor 120. The payment module 1040 is sent a scheduled payment. When the scheduled payment is received, the license will be extended. The FTP license file server 1010 receives new encrypted license. The control center 520 requests and receives the updated encrypted license file and determines that the encrypted license file has changed. The new encrypted license is stored inside the flash memory inside the intraoral sensor 120. The control center 520 periodically reads the encrypted license file from the FTP license file server 1010 and determines whether it has been updated compared to the license read from the intraoral sensor 120.

Referring still to FIG. 5 in conjunction with FIG. 4 the sensor licensing system 1000 provides live license control of the intraoral sensor 120 with no user interaction by contacting a server for an encrypted licensing code which incorporates date and image counter checking to prevent tampering. This allows the intraoral sensor 120 to be turned on/off remotely if a periodic subscription payment is not made. This requires that license checks be made requiring periodic connection of the intraoral sensor and computer to the internet. The sensor licensing system 1000 includes an on/off mechanism, a driver and a sensor. The on/off mechanism is coupled to the intraoral sensor and has an encrypted licensing code so that the on/off mechanism turns on and off the intraoral sensor. The driver processes the encrypted license code stored in the sensor memory and determines if license code is expired based on the computer clock, exposure counter or other desired limits. The sensor periodically checks an online server for an updated encrypted license code and stores new license code in sensor memory. The single control may be located on a plurality of individual computers and can handle licensing of multiple intraoral sensors 120. The updated license code is created and stored on the server when a new periodic subscription payment is made. The sensor periodically checks and downloads any updated license code. Failure to make a payment will result in no encrypted license code update being created and eventual expiration of the old encrypted license code. License updates are created upon receiving subscription renewal payments and the transfer and storage of the updated license files involves encryption of the license information to prevent tampering with the license file contents. Since the x-ray sensor subscription plans typically involves setting and maintaining an expiration date for the license then it is of utmost importance that the licensing supervision system in the control center continuously verifies that the “system time” is correct and not being tampered with. This involves checking the PC's real time clock against an external clock source (network time via NTP) as well as utilizing the x-ray sensors non-volatile memory to keep track of the last time the sensor was used. The primary concern is to make sure no one adjust the time backwards to extend the valid license period so the basis for the tamper protection is to verify that the system time is always moving forward and never jumps back.

Referring still further to FIG. 5 in conjunction with FIG. 4 the control center 520 includes a driver, software including dental image acquisition software, a monitor and a dose optimization tool. The driver controls operation of the intraoral sensor on a computer. The control center software operates on the computer not only in real time, but also in the background in order for the control center to control image acquisition and settings for the intraoral sensor so that it can be used with almost any dental imaging software on the market. The control center software acts as the primary driver for the intraoral sensor and interfaces with other imaging applications to acquire and serve up images for processing and display. The control center software interfaces with other applications via either an SDK driver or a TWAIN driver. The control center software can perform preprocessing on the image before transferring to an imaging application, including but not limited to noise filtering, sharpening, contrast enhancement or histogram equalization. The monitor checks performance of the intraoral sensor during use and notifies the user of problems. The dose optimization tool guides user to perform initial exposure measurements of an x-ray head for optimal signal level to minimize patient exposure to ionized radiation thereby insuring optimal image quality while insuring that the lowest possible x-ray generator settings to achieve diagnosable x-ray images. The control center 520 saves and stores recent images to allow for remote review and diagnosis of sensor issues during debugging. The control center provides integrated support which is live with built-in support tools operating in the background, independent of the imaging application. The control center monitors real time for software or sensor firmware updates.

The licensing management system for an intraoral sensor involves the handling of license distribution and license enforcement for intraoral x-ray sensors so that it is kept generic to the various integrations of x-ray sensor products into 3rd party imaging software applications. The license distribution and enforcement are centered round a “control center” application that runs as a background service. The primary function of the control center is to enforce and renew the licenses and to serve as a single point of integration for third party applications. Since the control center 520 is always required then it will can perform background tasks such as image pre-processing and supervision of the image exposure level without any manual intervention by the end-users. The control center 520 serves as a convenient tool to be used for troubleshooting any issues that are reported from the field.

Referring again to FIG. 3 a dose calibration tool guides user to perform initial measurement of each individual x-ray heads for optimal dose without exposing the patient to ionized radiation. The dose optimization tool also insures optimal image quality while ensuring that the lowest possible x-ray generator settings are set, avoiding overdosing of patients. The dose optimization tool further recommends ideal dose/time settings for use base on Adult/Child selections for different anatomic regions (like anterior, posterior) since the density of the bones and teeth varies accordingly. A real time dose monitor, after dose optimization, will continue to monitor exposure of every image and notify the operator if dose is too high or low immediately after images acquisition during clinical use. This is currently done by competitive imaging applications, but the subject dose optimization tool operates in the background thereby allowing it to be used independent of the imaging application being used. An exposure monitor counts sensor usage and records data such as total exposures and dose. A recent image storage system saves recent images to allow for remote review and diagnosis of sensor issues during debugging. An integrated support system includes live, built in support tools operating in the background, independent of the imaging application. The intraoral sensor receives updates and monitors real time for software or sensor firmware updates.

From the foregoing, it can be seen that a licensing management system for an intraoral sensor which manages licenses, licensing data, tamper protection, x-ray dose optimization, exposure monitoring and other information.

Accordingly, it is intended that the foregoing disclosure and showing made in the drawing shall be considered only as an illustration of the principle of the present invention.

What is claimed is:

1. A sensor licensing system for an intraoral sensor wherein said sensor licensing system comprises:
a. an on/off mechanism coupled to the intraoral sensor wherein said on/off mechanism has an encrypted licensing code whereby said on/off mechanism turns on and off the intraoral sensor;
b. a driver that processes the encrypted license code stored in the sensor memory and determines if license code is expired based on the computer clock, exposure counter or other desired limits; and
c. sensor periodically checks an online server for an updated encrypted license code and stores new license code in sensor memory.

2. A sensor licensing system for an intraoral sensor according to claim 1 wherein an updated license code is created and stored on the server when a new periodic subscription payment is made. The sensor periodically checks and downloads any updated license code. Failure to make a payment will result in no license code update being created and eventual expiration of the old license code.

3. A sensor licensing system for an intraoral sensor according to claim 2 said sensor licensing system requires periodic connection of the intraoral sensor and said computer to the internet for license checks.

4. A sensor licensing system for an intraoral sensor according to claim 2 wherein said encrypted licensing code incorporates date and image counter checking to prevent tampering.

5. A sensor licensing system for an intraoral sensor according to claim 4 wherein said sensor licensing system includes means of detecting potential expiration of sensor license and notifying user of need to either update license or connect sensor to a server to check/update licensing status.

6. A control center for an intraoral sensor comprises:
a. a driver that controls operation of the intraoral sensor on a computer; and
b. software which operates on said computer not only in real time, but also in the background so that said control center controls image acquisition and settings for the intraoral sensor so that it can be used with almost any dental imaging software on the market.

7. A control center for an intraoral sensor according to claim 6 wherein said control center also monitors performance of the intraoral sensor during use and notifies the user of problems.

8. A control center for an intraoral sensor according to claim 6 wherein dental image acquisition software acts as the primary driver for the intraoral sensor and interfaces with other imaging applications to acquire and serve up images for processing and display wherein said dental image acquisition software interfaces with other applications via either an SDK driver or a TWAIN driver whereby said dental image acquisition software can perform preprocessing on the image before transferring to an imaging application, including but not limited noise filtering, sharpening, contrast enhancement or histogram equalization.

9. A dose optimization tool for an intraoral sensor wherein said dose optimization tool guides user to perform initial exposure measurements of an x-ray head for optimal signal level to minimize patient exposure to ionized radiation thereby insuring optimal image quality while insuring that the lowest possible x-ray generator settings to achieve diagnosable x-ray images.

10. A dose optimization tool for an intraoral sensor according to claim 9 wherein said dose optimization tool recommends ideal dose/time settings for use based on adult/child and different x-ray energies.

11. A dose optimization tool for an intraoral sensor according to claim 10 wherein said dose optimization tool includes a real-time signal monitor which monitors image level and will continue to monitor exposure of every image and notify the operator if signal is too high or low immediately after images acquisition during clinical use.

12. A dose optimization tool for an intraoral sensor according to claim 11 wherein said dose monitor of said dose optimization tool operates in the background thereby allowing it to be used independent of the imaging application software being used.

13. A dose optimization tool for an intraoral sensor according to claim 11 wherein said exposure monitor of said dose optimization tool counts sensor usage and records data such as total exposures.

14. A control center for an intraoral sensor according to claim 7 wherein said control center further saves and stores recent images to allow for remote review and diagnosis of sensor issues during debugging.

15. A control center for an intraoral sensor according to claim 7 wherein said control center still further provides integrated support which is live with built-in support tools operating in the background, independent of the imaging application.

16. A control center for an intraoral sensor according to claim 7 wherein said control center also still further monitors real time for software or sensor firmware updates
